(19) United States
(12) Patent Application Publication
(10) Pub. No.: US 2004/0077593 A1
(43) Pub. Date: Apr. 22, 2004

(54) Numbing gel for hair removal

(76) Inventor: Susan M. Marron, Studio City, CA (US)

Correspondence Address: LITTMAN LAW OFFICES, LTD., P.O. BOX 15035 CRYSTAL CITY STATION, ARLINGTON, VA, 22215, US

(21) Appl. No.: 10/274,131

(22) Filed: Oct. 21, 2002

(51) Int. Cl. A61K 31/716; A61K 31/24

(52) U.S. Cl. 514/57; 514/536

(57) ABSTRACT

A numbing gel composition used in preparation for a body hair removal procedure such as the hot wax method. The gel composition contains lidocaine, tetracaine, methycellulose, and normal saline. The composition renders the skin substantially non-painful for the subsequent depilatory hot wax method.

### BACKGROUND OF THE INVENTION

1. Field of the Invention

The present invention relates generally to pain blocker compositions. More specifically, the invention is a topical anesthetic gel composition comprising lidocaine, tetracaine, methylcellulose, and normal saline solution for reducing pain associated with human body hair removal.

2. Description of the Related Art

The relevant art of interest describes various skin numbing compositions and related hot wax devices, but none discloses the present invention. There is a need for an effective, economical, topical, anesthetic composition for assisting in the field of human body hair removal. The related art of interest will be described in the order of perceived relevance to the present invention.

U.S. Pat. No. 5,585,398 issued on Dec. 17, 1996, and U.S. Pat. No. 5,942,543 issued on Aug. 24, 1999, to Amy A. Ernst describes a topical, anesthetic composition comprising lidocaine, adrenaline and tetracaine in either a gel base or an aqueous base, respectively. The compositions are distinguishable for requiring adrenaline.

U.S. Pat. No. 6,429,228 B1 issued on Aug. 6, 2002, to Toshio Inage et al. describes a local anesthetic composition for external use comprising lidocaine or prilocaine and salts thereof, a percutaneous absorption accelerator such as caprylic acid, ethanol and/or isopropyl alcohol, and water. The composition is distinguishable for requiring prilocaine and caprylic acid, and lacking lidocaine and tetracaine.

U.S. Pat. No. 4,883,654 issued on Nov. 28, 1989, to Deborah A. Young describes a cosmetic toiletry formulation in lotion or cream form for use prior to mechanical hair depletion such as eyebrow plucking. The composition contains menthol and eugenol. The composition is distinguishable for requiring menthol and eugenol, and lacking lidocaine and tetracaine.

U.S. Pat. No. 3,426,137 issued on Feb. 4, 1969, to Richard F. Philpitt et al. describes hair growth inhibiting compositions with substituted benzophenones. The compositions are distinguishable for requiring benzophenones and lacking lidocaine and tetracaine.

U.S. Pat. No. 4,720,489 issued on Jan. 19, 1988, to Douglas Shander describes a topical composition for hair growth modification for men containing an anti-androgen material and an ornithine decarboxylase inhibitor. The composition is distinguishable for requiring an anti-androgen material and an ornithine decarboxylase inhibitor.

U.S. Pat. No. 4,748,022 issued on May 31, 1988, to John A. Busciglio describes a topical composition for the treatment of pain and inflammation of lesions containing diphenhydramine hydrochloride, lidocaine hydrochloride, aloe vera, propolis, and sufficient base, i.e., aluminum hydroxide gel, to raise the pH to 8-9. The composition is distinguishable for requiring diphenhydramine hydrochloride, aloe vera, and propolis.

U.S. Pat. No. 4,830,633 issued on May 16, 1989, to Yutaka Hori et al. describes a depilation method including the coating of a depilatory agent containing an aqueous solution or emulsion of thiolglycolic acid or salt thereof, strontium sulfate, and sodium sulfide or potassium sulfide. The composition is distinguishable for requiring thioglycolic acid and numerous other substances.

U.S. Pat. No. 4,954,487 issued on Sep. 4, 1990, to Eugene R. Cooper et al. describes skin penetrating topical pharmaceutical compositions containing an active compound such as salicylic acid or ibuprofen, a first vehicle composition of a C₃-C₄ diol, diol ester or diol ether and a cell envelope-disordering compound such as methyl myristate. The compositions are distinguishable for requiring pain-alleviating compositions, diol compounds and methyl myristate.

U.S. Pat. No. 5,209,724 issued on May 11, 1993, to Avtar S. Dhaliwal et al. describes a composition containing an arginine compound as an inhibitor of nitric oxide synthetase to reduce mammalian hair growth. The composition is distinguishable for requiring an arginine compound.

U.S. Pat. No. 5,580,901 issued on Dec. 3, 1996, to David G. Boardman et al. describes a storage stable composition for topical or percutaneous anesthesia comprising an amethocaine free base, an aqueous gelling agent such as methylcellulose, and from 1 to 30% by weight of an alkali or an alkaline earth metal cation. The composition is distinguishable for requiring an amethocaine free base.

U.S. Pat. No. 6,291,478 B1 issued on Sep. 18, 2001, to Jean-Baptise Galey describes compositions for treating sensitive skins comprising compounds belonging to the benzylaminodiacetamide family having good anti-irritant and soothing properties. The compositions are distinguishable for requiring benzylaminodiacetamide compounds.

U.S. Pat. No. 6,329,398 B1 issued on Dec. 11, 2001, to Stephen M. Zappala describes a pharmacological agent for use as preemptive analgesia comprising 1% lidocaine hydrochloride, 0.25% bupivacaine hydrochloride in a ratio equal to or less than 10:1. The composition is distinguishable for requiring bupivacaine hydrochloride.

U.S. Pat. No. 4,773,784 issued on Sep. 27, 1988, and U.S. Pat. No. 4,958,951 issued on Sep. 25, 1990, to Samuel J. Mann describes a hot wax hair remover system for heating and applying depilatory to skin for removal of hair. The systems are distinguishable for being based on devices used for hot wax removal of hair.

European Patent Application No. 0 348 862 A2 published on Jan. 3, 1990, for Alexander Levin et al. describes an auxiliary skin cooling device for a hair-removing apparatus comprising an internal cooling element which has a heat conducting surface capable of cooling the skin prior to removal of hair by the hair-removing unit of the apparatus. The cooling of the skin substantially reduces the pain usually associated with hair removal by such an apparatus. The hair-removing apparatus is distinguishable for requiring a cooling device and lacking any chemical application.

None of the above inventions and patents, taken either singularly or in combination, is seen to describe the instant invention as claimed. Thus, a numbing gel for hair removal solving the aforementioned problems is desired.

### SUMMARY OF THE INVENTION

The present invention is directed to a pain blocker composition useful for preparing the body skin for wax or the like depilatory operation. More specifically, the invention is a topical anesthetic gel composition comprising lidocaine, tetracaine, methylcellulose, and normal saline. This composition effectively anesthetizes the affected skin area to neutralize any pain resulting from wax removal of hair.

Accordingly, it is a principal object of the invention to provide a topical anesthetic composition according to the present invention.

It is another object of the invention to provide a topical anesthetic composition for neutralizing any pain associated with the wax removal of body hair.

It is a further object of the invention to provide a topical anesthetic composition in gel form according to the present invention.

Still another object of the invention is to provide a topical anesthetic composition containing lidocaine, tetracaine, methycellulose, and normal aqueous saline solution according to the present invention.

It is an object of the invention to provide improved elements and arrangements thereof for the purposes described which is inexpensive, dependable and fully effective in accomplishing its intended purposes.

### DETAILED DESCRIPTION OF THE PREFERRED EMBODIMENT

The present invention is directed to a human body hair removal aid composition useful in the preparation for depilatory procedures such as the hot wax process. It has been found that minimal or no skin pain is observed when the numbing composition is used before the hot wax hair removal procedure. Moreover, no allergic skin reaction has been observed using this innovative preparatory composition. It is known in a hospital emergency room that a composition containing lidocaine, tetracaine and epinephrine are used for dulling external body pain. However, epinephrine is unnecessary in this composition. Methyl cellulose is added to create a gel base. If the composition is applied to the skin, and allowed to be absorbed by the skin for 10 to 15 minutes, one can proceed with the waxing process to remove the treated hair without any or little pain attached to the physical removal of the undesired hair. The depilatory effect can last up to three months. Up to now, most women prefer other means than hot wax removal, because the process is so painful.

The innovative composition contains 2-6 wt. % lidocaine, 0.3-0.7 wt. % by weight tetracaine, 0.6-1.2 wt. % normal saline in aqueous solution, and the balance of 92.1-97.1 wt. % methylcellulose gel having a viscosity of 300-500 centipoises at room temperature. A specific preferred composition contains 4 wt. % lidocaine, 0.5 wt. % tetracaine, 0.9 wt. % Normal saline in aqueous solution, and the balance of 94.6 wt. % methylcellulose gel having a viscosity of 400 centipoises at room temperature. It should be understood that one Normal solution of sodium chloride in water would be one equivalent of sodium chloride in weight per liter of water or 28 grams of sodium chloride in 1000 ml. of water. Therefore, 0.9 wt. % of a Normal saline solution will be 25.2 grams of sodium chloride per liter of the innovative composition.

This composition is applied to the hair and skin, remains in contact for 10-15 minutes for maximum absorption by the skin and hair, and dried. Then, the conventional waxing process can begin to actually remove the treated hair. It has been established that little or no pain results from the waxing process to remove the treated hair from one's face, legs, arms, and the like. The conventional waxing process normally taken every several months is painful, and most women avoid it because of the resulting pain. It should be noted that the pain involved with individual plucking of body hairs by the person would also be eliminated by the use of this composition.

Thus, a chemical composition has been found which substantially eliminates the pain normally encountered when the waxing process is utilized.

It is to be understood that the present invention is not limited to the embodiment described above, but encompasses any and all embodiments within the scope of the following claims.

I claim:

1. A numbing gel composition for hair removal consisting essentially of:
lidocaine;
tetracaine;
normal saline solution; and
methylcellulose gel.

2. The numbing gel composition according to claim 1, wherein 2 to 6 wt. % lidocaine is present.

3. The numbing gel composition according to claim 1, wherein 0.3 to 0.7 wt. % tetracaine is present.

4. The numbing gel composition according to claim 1, wherein 0.6 to 1.2 wt. % Normal saline solution is present.

5. The numbing gel composition according to claim 1, wherein methylcellulose having a viscosity of 300-500 centipoises is present in a major amount.

6. The numbing gel composition according to claim 1, wherein 4 wt. % lidocaine is present.

7. The numbing gel composition according to claim 1, wherein 0.5 wt. % tetracaine is present.

8. The numbing gel composition according to claim 1, wherein 0.9 wt. % Normal saline solution is present.

9. The numbing gel composition according to claim 1, wherein methylcellulose having a viscosity of 400 centipoises is present in a major amount.

10. A numbing gel composition for hair removal consisting essentially of:
2-6 wt. % lidocaine;
0.3-0.7 wt. % tetracaine;
0.6 to 1.2 wt. % Normal saline solution; and
the balance being methylcellulose gel having a viscosity of 300 to 500 centipoises.

11. A numbing gel composition for hair removal consisting essentially of:
4 wt. % lidocaine;
0.5 wt. % tetracaine;
0.9 wt. % Normal saline solution; and
the balance being methylcellulose gel having a viscosity of 400 centipoises.
